(19) United States
(12) Patent Application Publication
(10) Pub. No.: US 2016/0296457 A1
(43) Pub. Date: Oct. 13, 2016

(54) Topical Composition Precursor for Skin Treatment Compositions.

(71) Applicant: David Paul Dawson, Coventry, RI (US)

(72) Inventor: David Paul Dawson, Coventry, RI (US)

(21) Appl. No.: 14/681,840

(22) Filed: Apr. 8, 2015

(51) Int. Cl. A61K 8/81; A61Q 19/00; A61K 8/73

(52) U.S. Cl. CPC A61K 8/8176; A61K 8/731; A61Q 19/007; A61K 2800/20

(57) ABSTRACT

The invention when incorporated in skin treatment compositions provides the intended skin treatment compositions to be retained longer on the skin and/or to feel more pronounced and/or enhanced to the skin and works in compositions that are intended to be applied on wet skin and works in compositions that are intended to be applied on dry skin. The invention applies to (a) oil in water emulsion compositions; (b) water in oil emulsion compositions; (c) alcohol and water compositions; (d) aqueous compositions; (e) silicone and water compositions and (f) any combination thereof. The invention is compatible across product lines such as soaps, lotions and alcohol/water gel hand sanitizers in which soaps and lotions compliment one another, soaps and alcohol/water gel hand sanitizers compliment one another and lotions and alcohol/water gel hand sanitizers compliment one another to provide the benefits of the ingredients that are intended to aid, nourish and protect the skin. The invention allows compositions to impart more desirable benefits to skin as well as adds customer appeal for the product to which it has been added or incorporated.

### FIELD OF THE INVENTION

The present invention relates to the treatment, enhancement and/or duration of compositions that are designed for use on or to the skin. By adding or using this invention during the manufacturing of soaps, lotions, creams, ointments, gels, skin sanitizers and other types of skin treatment compositions, it enhances the intended benefit of the composition to the skin such as making the composition feeling more pronounced and extends the duration of the intended benefit of the composition to the skin thus having a longer lasting effect of the composition on or to the skin. Thus, the consumer obtains more of the intended benefit of the skin treatment compositions.

### BACKGROUND OF INVENTION

Naturally occurring and synthetic polymers and celluloses have been used in pharmaceutical and cosmetic preparations for several decades which include hydrophilic polymers, hydrophilic cellulose, hydrophobic polymers, and polymers having hydrophilic and hydrophobic properties. Hydrophilic polymers and hydrophilic cellulose are often used as thickeners and/or film formers. Hydrophobic polymers are often used because of their ability to hold active ingredients and to bind to skin. This invention provides for a composition of hydrophilic polymers and hydrophilic celluloses that provide for the similar abilities of the hydrophobic polymers which is ability to hold active ingredients and to bind them to skin in a aqueous composition rather than have to resort to to a non-aqueous hydrophobic polymer type composition.

There are many, almost limitless compositions that are designed to be used on and to the skin for one purpose or another. The makers of soaps, lotions, creams, ointments, gels, skin sanitizers and other types of skin treatment compositions are constantly looking for methods, additives and compositions to be used in their product compositions to reduce costs and to increase the appeal and performance of their products and compositions. By using or adding variations of this invention to the many current and future skin treatment compositions, some of the more expensive ingredients may be reduced and still achieve similar results. By using or adding variations of this invention during the manufacturing of the many current and future skin treatment compositions, the appeal and performance of the intended benefits of the skin treatment compositions will likely be increased. Depending upon the nature of the composition to which this invention is used in or added to, some changes in the manufacturing process of the skin treatment compositions will most likely be needed to accommodate the invention.

Using soap or skin cleansers as an example, Soap's and cleanser's intended purpose for the most part is to cleanse the skin. Most realize that soaping and cleansing remove some skin lipids also known as skin oil and most realize that soaping and cleansing the skin will dry out the skin and remove some skin nutrients. Because soaps and skin cleansers do this to the skin, manufactures are consistently looking for efficient and cost effective ways of replacing and replenishing those skin essential lipids and oils that are removed during the cleansing of the skin and to provide the consumer with a skin healthy product as well of having the consumer experience a healthy skin feel.

Humectants are often added to many soap and skin cleanser compositions to moisturize the skin. Emollients are often added to many soap and skin cleanser compositions to soften the skin. Antioxidants are sometimes added to soap and skin cleanser compositions to prevent oxidation which causes free radicals to occur thus damage to the skin results. Lipids or oils, (as an additive) are often added to many soap and skin cleanser compositions to replace or nourish the skin lipid levels which are partly removed by cleansing. These are just a few of the widely known and used soap and skin cleanser additives, ingredients and compositions. There are many more additives, ingredients and compositions than are listed here. By incorporating this invention in soap and cleansers during the manufacturing process, the invention enhancers and extends the intended benefits of the additives. This means for example that the intended moisturizing effect of the humectants is more pronounced than without the use or addition of the invention and the moisturizing feel lasts longer than without the use or addition of the invention. The same would be true for another example that the intended skin softening effect of the emollients is more pronounced than without the use or addition of the invention and the soft skin feel lasts longer than without the use or addition of the invention. In addition, as a result of adding or incorporating the invention into these compositions, it aids and/or provides in achieving an adequate level of the additives, ingredients and/or compositions which are intended to benefit the skin are retained on the skin after rinsing and/or towel drying.

During the initial trial of the invention used in premium bar soap, premium bar shampoo, premium liquid hand soap, national brand dishwashing liquid, premium hand and skin lotion and premium butter creams, all the participants indicated a significant overall approval experience and an overall increase in appeal for the compositions which contained the invention verses the compositions that did not. Because the invention is relatively simple to incorporate into most of these compositions and because the initial results are predominantly based upon personal preference & personal past experiences with available over the counter skin treatment compositions, it became clear early on that the invention had succeeded in it's objective to enhance, improve and extend the benefits of the skin care compositions.

In the initial trial, each of the products listed in the above paragraph, 3 different variations of each product was prepared. The different soaps and lotions that were used in the trial are those premium products sold in bulk which are expected to re-sold and re-marketed under a new name or new brand sometimes referred to a “house label” or “private label”. The applicant sort premium products to test with as a precaution to avert the perception of taking a in-expensive product, adding a few extra ingredients and calling it a invention. The 3 prepared variations were as follows:
1. The original product as purchased.
2. The original product with added glycerin as the humectant and with an added butter or essential oil as the emollient and with additional emulsifiers to incorporate the added ingredients.
3. A preparation of #2 above plus the invention plus emulsifiers to incorporate all the ingredients.

The participants chosen suffered from a mild form of eczema with dry chapped hands, a few had minor split skin. The participants were from the medical field whose skin was exposed to repeated use of alcohol/water blend hand sanitizers. The participants agreed that the #1 premium products were very nice and felt and acted like a premium product should. The participants also agreed that the premium products with the added humectant (moisturizer) and added emollient (skin softener) as prepared in #2 felt and functioned even nicer than the original #1 products. It was the #3 invention preparation that the participants described that they experienced the “wow” factor. The participants indicated the #3 invention preparation gave them the most premium feel they have ever experienced. The longest lasting as well. Many of the participants indicated several days would pass before the premium rich feeling would dissipate away. In addition, several participants decline to lotion their hands after using the soaps citing that their hands felt so good, so nourish and so moist that they just didn't feel lotion was necessary and declined after just a few soap washings.

As a result of the successful initial trial, a smaller subsequence trial very similar to the initial trial was conducted. In this trial, the ingredients Allantoin and DL Panthenol were added to preparation #2 and #3 and the participants chosen suffered from a more moderate form of dermatitis and eczema and a mild for of psoriasis. Also, only premium bar soap, premium bar shampoo, premium liquid hand soap, national brand dishwashing liquid were used in this smaller subsequence trial since the participants from the earlier initial trial declined to use lotion because of the rapid improvement to their skin. The object of this trial was to see if participants who with the more moderate skin disorder would be able to comfortable go between washing and cleansings of their hands and faces without the desire to use a lotion. As with the initial trial, the #2 preparation (without the invention) would dissipate within a few hours and the participants expressed a desire to use a lotion to comfort their hands between washings. The participants indicated the #3 invention preparation gave them a rich long lasting premium feel, relieved the itching, soreness associated with their skin disordered and gave them relief extending through to their next washing, cleansing or bathing.

The 3 subsequence trial prepared variations were as follows:
1. Not Used—The original product as purchased used in the subsequence trial of moderate form of dermatitis and eczema participants.
2. The original product with added glycerin as the humectant, with an added butter or essential oil as the emollient, with DL Panthenol to reduce redness, to act as a anti-inflammatory agent and to reduce acne on the face, with Allantoin to promote skin growth and with additional emulsifiers to incorporate the added ingredients.
3. A preparation of #2 above plus the invention plus emulsifiers to incorporate all the ingredients.

The one participant who suffered from mild psoriasis continually used daily the #3 invention soap preparation and as of the 12th week, around the time of the writing of this patent application, had cleared all visual signs of the participants Psoriasis over entire body except for one small areas around participants right knee. That particular participants trial is still ongoing as of the writing of this patent application.

### SUMMARY OF THE INVENTION

What is new and unique about the invention is its compatibility to be incorporated into 4 of the 5 types of emulsion compositions. It is compatible with “oil in water”, “water in oil”, “alcohol and water” and “aqueous” emulsion compositions and combinations thereof. It is not compatible with the 5th type “all oil” type emulsion compositions also referred to as “anhydrous” compositions. A substance is said to be Anhydrous if it contains no water. Also, the invention is compatible with a variety of hybrid compositions such as oil, water and silicone compositions.

The invention is new and unique because it is particularly useful, extremely efficient, cost effective and convenient when the invention is incorporated into in a variety of soaps and cleansers. The reason being by adding a few typical lotion ingredients, such as antioxidants, emollients, humectants and vitamins to the soap or cleanser composition and add the invention into the composition, the purchase, use and repeated application of lotions may be eliminated in with consumers suffering from less than severe skin disorders such as eczema, dermatitis and mild psoriasis. Also, many consumers forget to lotion their skin disorders immediately following cleansing as they usually have a short period of comfort as a result of soothing ingredients contained in their soap and cleanser products. By the time the soothing effect dissipates after cleansing, the consumer is not likely to be near their lotion supply and may have to suffer with itching and soreness until they can get back to their lotion supply of which is usually located in the home or away from where they are at the moment. The subsequence trial clearly indicated the participants in #3 invention preparation gave them a rich long lasting premium feel, relieved the itching, soreness and irritation associated with their skin disordered and gave them relief extending through to their next cleansing without lotion in between cleansings.

Alcohol/water gel hand sanitizers is a must for the vast majority who work in the medical field, nursing facilities, barbers and hair dressers to name a few. The frequent and repetitive use of Alcohol/water gel hand sanitizers even those with moisturizers can cause eczema and contact dermatitis. Heavy users of the Alcohol/water gel hand sanitizers are at the most risk for eczema and contact dermatitis. Due to regulations, cutting back on heavy use of Alcohol/water gel hand sanitizers is nearly impossible. In most cases, regulations require medical workers such as nurses and medical technicians to hand sanitize when entering and again when leaving a patients room. A busy nurse scurrying between patient rooms on a busy day is repeatedly using hand sanitizers sometimes 10 to 15 times an hour. That much Alcohol/water gel causes the skin to dry out and removes some of the skin lipids or skin oils from the skin causing the skin to split, crack and/or peel. As a result, the skin becomes sore, irritated and painful. Lotions can be used but very often the lotion's moisturizers and lipids are easily diluted and removed by the continued use of the Alcohol/water gel sanitizer.

Thus, there remains a need for an invention which is compatible with soaps and cleansers, and with skin lotions and creams and with Alcohol/water blend gel hand sanitizers so that one product or composition does not wash off, remove or dilute another product or compositions intended beneficial ingredient(s) very easily. The applicant's invention fulfills that need. Using, adding and/or incorporating the invention into a line of soaps, lotions and Alcohol/water gel hand sanitizers will fulfill the gaps that current soap, lotions and Alcohol/water gel hand sanitizers do not currently have. The invention will allow the vast majority who work in the medical field, nursing facilities, barbers and hair dressers (just to name a few) who rely on and are required to use Alcohol/water blend gel hand sanitizers to be able to use the inventers composition in soap that will not wash off very easily the inventers composition in lotion and be able to use repeatedly the inventers composition in a alcohol/water blend gel hand sanitizer that will not wash off very easily the inventers composition in lotion.

Putting it in simple terms, there is no known composition that is compatible with “oil in water”, “water in oil”, “alcohol and water” and “aqueous” emulsion compositions that work in harmony with one another across product lines such as soaps, lotions and alcohol/water gel hand sanitizers in which soaps and lotions compliment one another, in which soap and alcohol/water gel hand sanitizers compliment one another and in which lotions and alcohol/water gel hand sanitizers compliment one another to provide the benefits of the ingredients that are intended to aid, nourish and protect the skin.

A further objective of the invention when used in soaps, lotions and alcohol/water gel hand sanitizers (to name a few) is to provide the consumer with a compatible line of products that is perceived to moisturize, nourish, sanitize, medicate and protect the skin while still being perceived to leave the skin clean and with a natural look and a moisturized non-greasy look and feel.

### DESCRIPTION OF THE INVENTION

The subject invention provides skin care treatment compositions that are designed for skin soaps and cleanses, skin creams, skin lotions, skin ointments, skin gels, skin sanitizers and skin treatment compositions a method by which desired ingredients may be delivered to the skin and retained by the skin in a more efficient manor across these various types of compositions.

More specifically, the invention provides a aqueous composition, comprising of water, a water-soluble polymer comprising of Polyvinyl-pyrrolidone, and a water-soluble cellulose groups comprising of carboxymethyl cellulose. This invention provides for a composition of hydrophilic polymers and hydrophilic celluloses that provide for the similar abilities of the hydrophobic polymers which is ability to hold active ingredients and to bind them to skin in a aqueous composition rather than have to resort to to a non-aqueous hydrophobic polymer type composition. This invention also provides when incorporated into compositions which contain both hydrophobic and hydrophilic polymers and/or celluloses provides additional holding and binding properties of desired ingredients to the skin.

The invention claimed is:

1. A topical composition precursor comprising of (a) a water-soluble polymer comprising of Polyvinyl-pyrrolidone; (b) a cellulose derivative with carboxymethyl groups comprising of carboxymethyl cellulose; (c) water.

2. A topical composition precursor according to claim 1, wherein the Polyvinyl-pyrrolidone is a water-soluble polymer made from monomer N-vinylpyrrolidone.

3. A topical composition precursor according to claim 1, wherein the carboxymethyl cellulose is a water soluble cellulose.

4. A topical composition precursor according to claim 1, wherein the water-soluble polymer Polyvinyl-pyrrolidone to water soluble cellulose carboxymethyl cellulose is provided at a ratio of about 1:2 to about 6:1 herein referred to as the polymer-cellulose composition.

5. A topical composition precursor according to claim 1, wherein the polymer-cellulose composition from claim 1 is dissolved in water to a ratio of about 1 part polymer-cellulose composition to 50-5000 parts water which is herein referred to as the precursor composition.
